(19) United States
(12) Patent Application Publication
(10) Pub. No.: US 2011/0046395 A1
(43) Pub. Date: Feb. 24, 2011

(54) PROCESS FOR THE MANUFACTURE OF A 6-FLUORO-1,2-DIHYDRO-2-OXO-3H-INDOL-3-YLIDENE DERIVATIVE

(75) Inventors: Werner Rall, Mittelbiberach (DE); Waldemar Pfrengle, Biberach (DE); Juergen Schnaubelt, Warthausen (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/863,502

(22) PCT Filed: Jan. 22, 2009
(86) PCT No.: PCT/EP09/00377

§ 371 Date: Nov. 8, 2010

(30) Foreign Application Priority Data
Jan. 25, 2008 (EP) 08150661.0

(51) Int. Cl. C07D 209/34

(52) U.S. Cl. 548/486

(57) ABSTRACT

The present invention relates to a process for the manufacture of the compound 4-[(Z)-[[4-[(dimethylamino)methyl]phenyl]amino](6-fluoro-1,2-dihydro-2-oxo-3H-in-dol-3-ylidene)methyl]-benzenepropanoic acid and to a new intermediate for the synthesis.

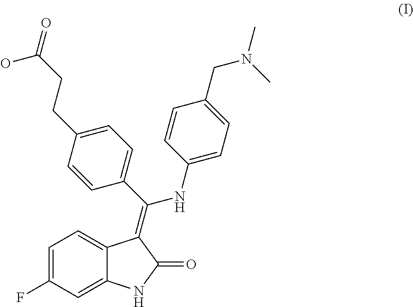

### BACKGROUND TO THE INVENTION

The present invention relates a process for the manufacture of the compound 4-[(Z)-[[4-[(dimethylamino)methyl]phenyl]amino-](6-fluoro-1,2-dihydro-2-oxo-3H-indol-3-ylidene)methyl]-benzenepropanoic acid and to a new intermediate for the synthesis.

The chemical formula of the compound 4-[(Z)-[[4-[(dimethylamino)methyl]phenyl]amino](6-fluoro-1,2-dihydro-2-oxo-3H-indol-3-ylidene)methyl]-benzenepropanoic acid is depicted below as Formula I.

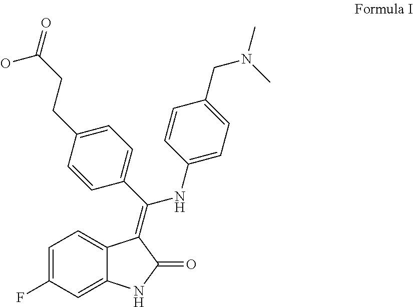

A number of 2-indolinone derivatives are already known in the prior art. Thus, for example, International Patent Applications WO 01/27081, WO 04/009546 and WO 04/009547 disclose 2-indolinone derivatives which have valuable pharmacological properties.

The compound of above formula I is disclosed in WO 04/009546 and WO 04/009547. In WO 04/009547, it is disclosed as example 10.1, however using a different nomenclature, namely 3-Z-[1-(4-dimethylaminomethylanilino)-1-(4-(2-carboxyethyl)phenyl)methylene]-6-fluoro-2-indolinone.

A process for the manufacturing of this compound is disclosed in WO 04/009547, under Example 10.1 via the procedure described in Examples 6.0, 5.1, 1.0 and using the staring material VI.22. However, in the manufacturing process disclosed in the prior art, the compound is synthesized using a complex procedure. Furthermore, the process described in WO 04/009547 uses reagents which are extremely toxic or explosive, and thus not really suitable for an up-scaling of the manufacture to a production in large amounts. For example, the reagents 1-hydroxy-1H-benzotriazol (HOBt) and O-benzotriazol-1-yl-N,N,N′,N′-tetramethyluronium-tetrafluoroborat (TBTU) are used in the process described in WO 04/009547, and both are coupling reagents with explosive properties. Known alternative reagents such as triphenylphosphine/carbon tetrachloride are, on the other hand very toxic. Thus, there may further be a danger in using the manufacturing process disclosed in WO 04/009547 for an up-scaling of the manufacture to a production in large amounts. Furthermore, the trimethyloxoniumtetrafluoroborate used for the alkylation of the hydroxymethyl group in WO 04/009547 is an expensive reagent and not available in larger amounts for a production process.

Like the 2-indolinone derivatives mentioned in the prior art, the compound of above Formula I also has, in particular, an inhibiting effect on various kinases, particularly receptor tyrosine kinases such as VEGFR1, VEGFR2, VEGFR3, PDGFRα, PDGFRβ, FGFR1, FGFR3, EGFR, HER2, c-Kit, IGFIR, Flt-3 and HGFR, and on the proliferation of cultivated human cells, particularly endothelial cells, e.g. in angiogenesis, but also on the proliferation of other cells, particularly tumour cells.

The pharmacologically valuable properties of the indolinone derivatives disclosed in the prior art and mentioned above constitute the basic prerequisite for an effective use of these compounds in pharmaceutical compositions. An active substance must in any case satisfy additional requirements in order to be manufactured in large scale and accepted for use as a drug. These requirements are a short, safe and not too expensive manufacturing process.

The problem underlying the present invention is thus the provision of a pharmaceutically active substance which is not only characterised by high pharmacological potency but also satisfies the above-mentioned requirements for its manufacture.

### SUMMARY OF THE INVENTION

This problem is solved by the manufacturing process and the new intermediate in accordance with the present invention.

A first object of the present invention is thus a process for the manufacture of the compound 4-[(Z)-[[4-(dimethylamino)methyl]phenyl]amino](6-fluoro-1,2-dihydro-2-oxo-3H-indol-3-ylidene)methyl]-benzenepropanoic acid, process which is described hereafter and depicted in the synthesis schemes below.

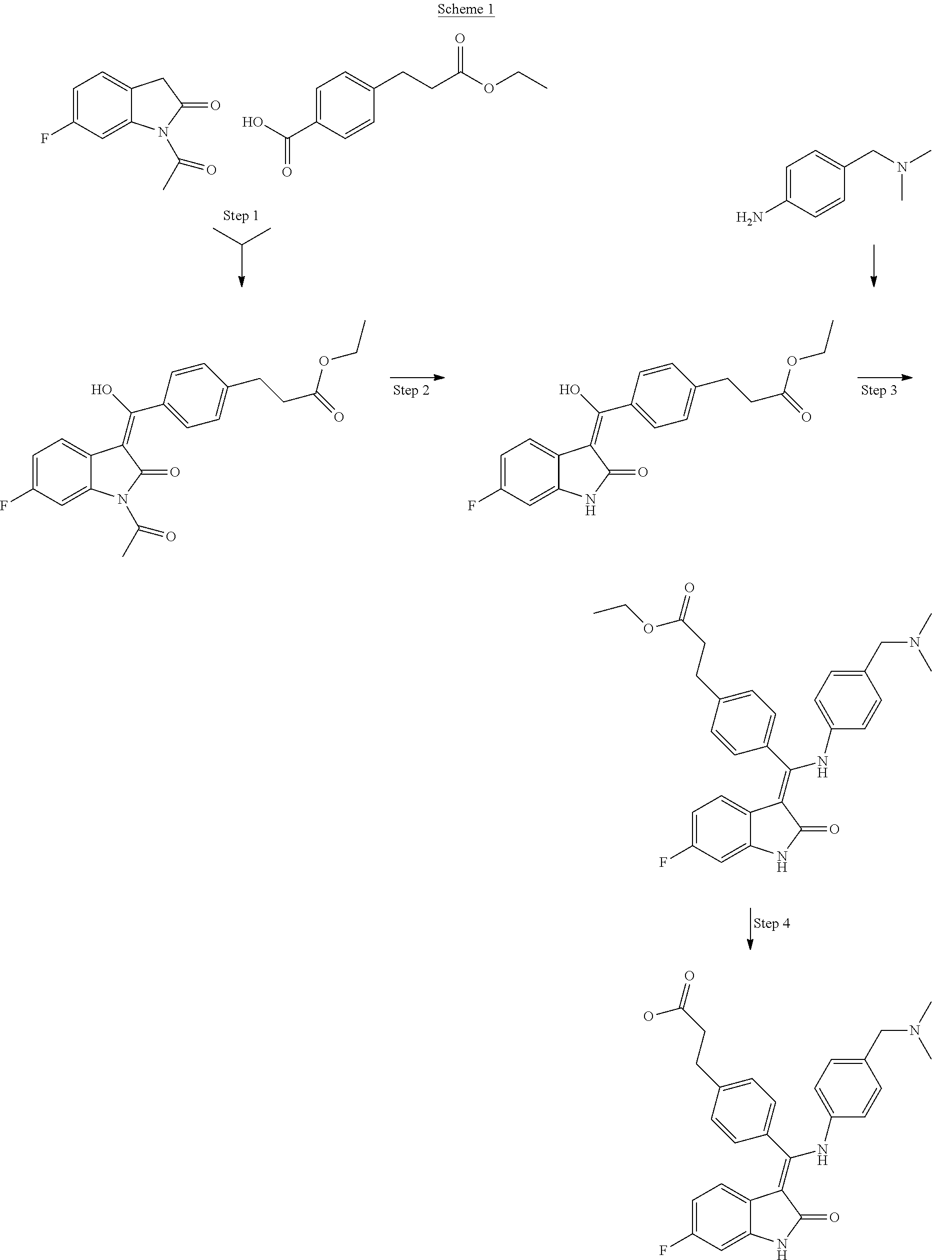

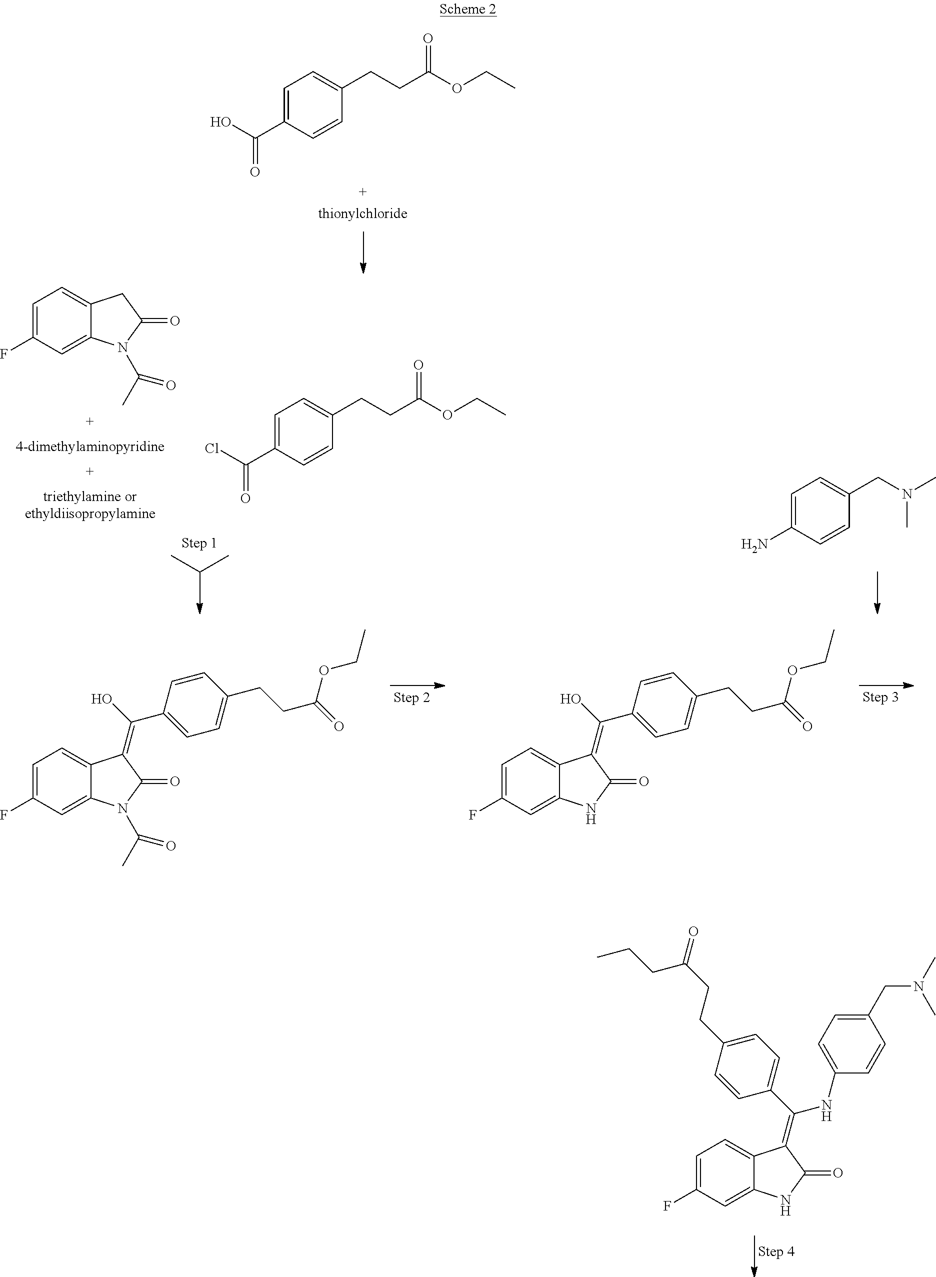

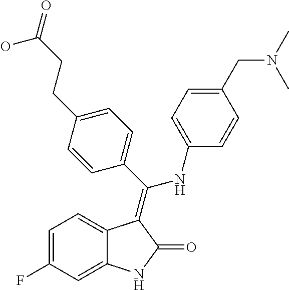

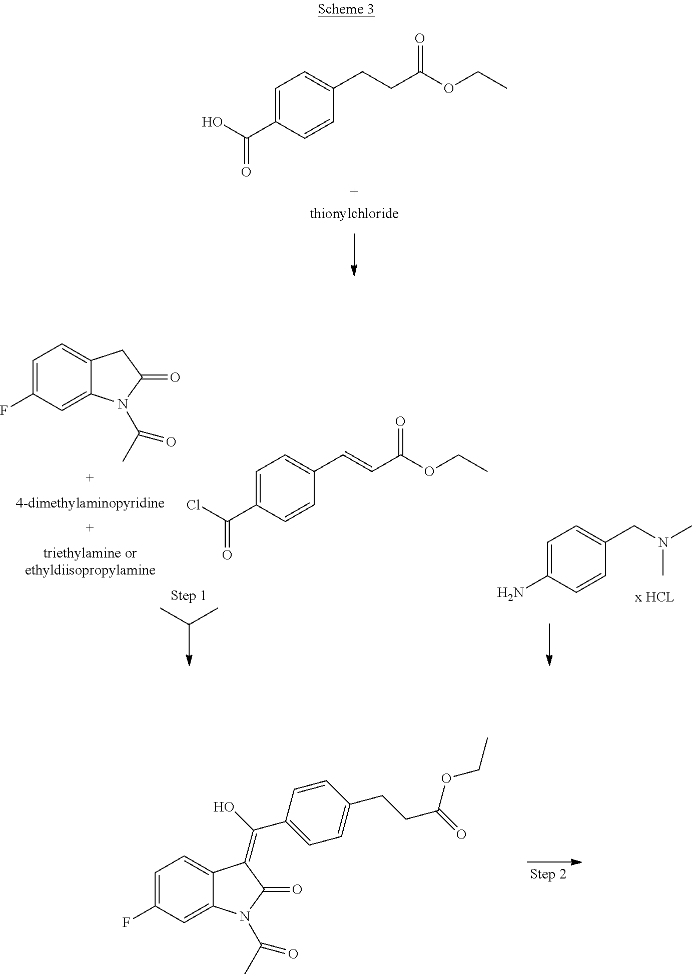

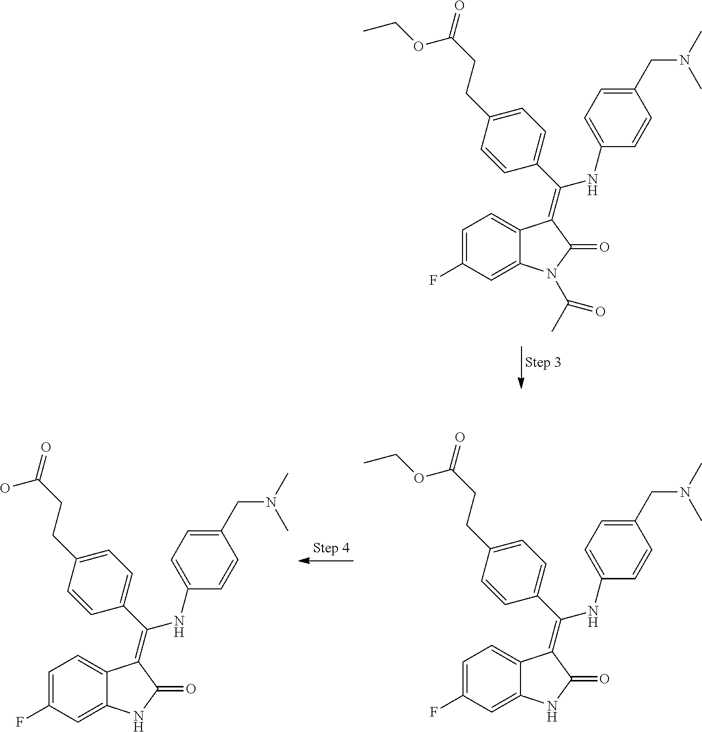

Thus, a first object of the present invention is a process for preparing the compound 4-[(Z)-[[4-[(dimethylamino)methyl]phenyl]amino](6-fluoro-1,2-dihydro-2-oxo-3H-indol-3-ylidene)methyl]-benzenepropanoic acid, as represented below as Formula I

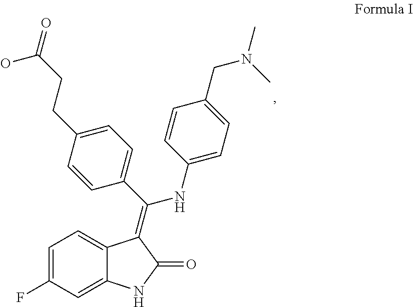

said process comprising the steps of
(a) reacting a compound of formula

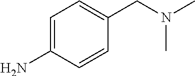

with
(i) a compound of formula

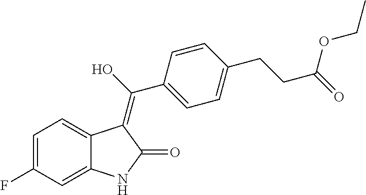

or with
(ii) a compound of formula

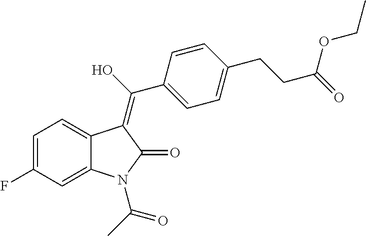

and
(b) subsequent de-esterification of the propanoic acid, ethyl ester group,
   wherein the removal of the acetyl group bound to the lactame group in the compound of formula

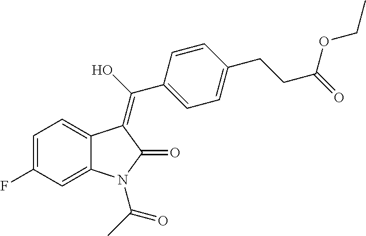

in reaction (ii) is performed after step (a),
and in which the reaction (a)(i) or (a)(ii) is performed in the presence of a mixture of reagents and solvents selected from:
Hexamethyldisilazane and p-toluenesulfonic acid monohydrate in the presence of triethylamine;
Hexamethyldisilazane and p-toluenesulfonic acid monohydrate in the presence of pyridine;
Hexamethyldisilazane and benzenesulfonic acid in the presence of triethylamine;
Hexamethyldisilazane and benzenesulfonic acid in the presence of pyridine;
Hexamethyldisilazane and trimethylsilylchloride;
N,O-bis(trimethylsily)acetamide and pyridine;
Trimethylsilylimidazolide and pyridine.
Thus, the reagents which may be used for the above processes (a)(i) or (a)(ii) are hexamethyldisilazane, trimethylsilylchloride, p-toluenesulfonic acid monohydrate or benzenesulfonic acid in the presence of triethylamine or pyridine, N,O-bis(trimethylsily)acetamide and pyridine, and trimethylsilylimidazolide and pyridine.
The solvents which may be used for the processes (a)(i) or (a)(ii) are hexamethyldisilazane, 1,4-dioxane, tetrahydrofurane, methyl-tetrahydrofurane, dimethylformamide, 1-methyl-2-pyrrolidinone, toluene.
Hence, hexamethyldisilazane may be used as well as reagent and as solvent, or both.
In a preferred embodiment, hexamethyldisilazane and trimethylsilylchloride may be used as reagent.
In a further preferred embodiment, hexamethyldisilazane or dioxane may be used as solvent.
The following illustrative conditions may thus be used.
   Mixture reagent/solvent: hexamethyldisilazane and dioxane
   Reaction temperature: 80-110° C.
   Reaction time: 60-70 hours

A further object of the present invention is the above process, wherein in step (a)(i) the compound of formula

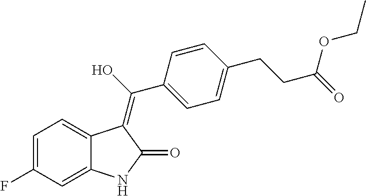

is obtained by removal of the acetyl group bound to the lactame group in a compound of formula

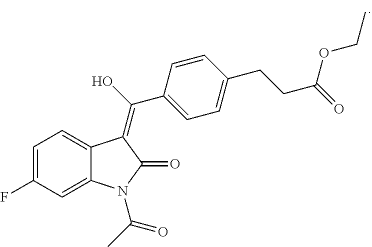

In a further embodiment in accordance with the present invention, the removal of the acetyl group from the lactame group in step (a)(i) is performed in the presence of sodium methoxide.
The following illustrative procedures and conditions may be used for this purpose.
   (1) Solvent/reagent: methanol with 1 equivalent sodium methoxide
      Reaction temperature: 30-60° C., preferably 60° C.
      Reaction time: 2 hours
   (2) Solvent/reagent: methanol with 0.17 equivalents Iodine
      Reaction temperature: 50-60° C.
      Reaction time: 4 Stunden
In a preferred embodiment, a mixture of methanol and sodium methoxide may be used.

A further object of the present invention is the above process, wherein the removal of the acetyl group from the lactame group in the compound of formula

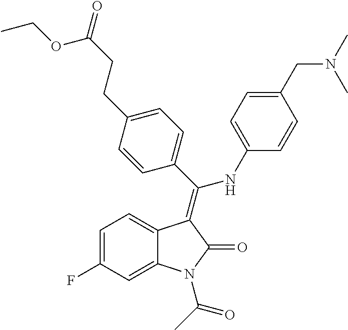

is performed by subsequent addition of methanol and sodium methoxide in the reaction medium of step (a)(ii).
In a further embodiment in accordance with the present invention, the removal of the acetyl group from the lactame group in the reaction medium of step (a)(ii) is performed in the presence of sodium methoxide.
The following illustrative procedure and conditions may be used for this purpose.
   (1) Solvent/reagent: methanol with 1 equivalent sodium methoxide
      Reaction temperature: 30-60° C., preferably 60° C.
      Reaction time: 2 hours
Alternatively, this process step may be performed in accordance with the following procedure, in which a solution of hydrochloride acid in ethanol is added to the reaction medium of step (a)(ii) at room temperature.
In a preferred embodiment, a mixture of methanol and sodium methoxide may be used.

A further object of the present invention is the above process, wherein the de-esterification of the propanoic acid, ethyl ester is performed in the same reaction medium as used for the removal of the acetyl group from the lactame group.

A further object of the present invention is the above process, wherein the removal of the acetyl group from the lactame group and the de-esterification of the propanoic acid, ethyl ester is performed in the same reaction medium.
In a preferred embodiment, a mixture of methanol/water and sodium hydroxide may be used as reaction medium.

A further object of the present invention is the above process, wherein the compound of formula

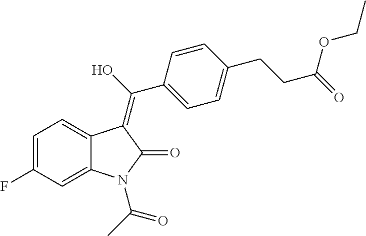

is obtained by reacting a compound of formula

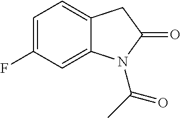

with the product of the reaction of a compound of formula

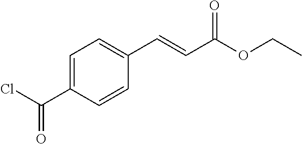

with 4-dimethylaminopyridine and triethylamine or with 4-dimethylaminopyridine and ethyldiisopropylamine. This step is shown in synthesis schemes 2 and 3.

The solvents which may be used for this process step are: dichloromethane, toluene, dimethylformamide or 1-methyl-2-pyrrolidinone, preferably dichloromethane.

A further object of the present invention is the above process, wherein the de-esterification of the propanoic acid, ethyl ester is performed, as shown in Step 4 of the synthesis schemes 1 to 3, by hydrolysis of the ester of the compound of formula

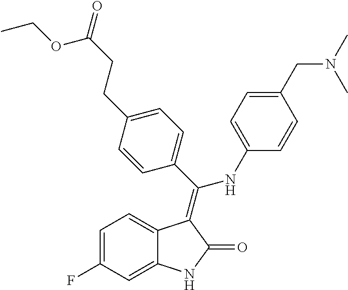

in the presence of sodium hydroxide.
The following illustrative procedure and conditions may be used for this purpose.
   (1) Solvent: mixture of EtOH/water, MeOH/water or tetrahydrofurane/water, preferably ethanol/water
      Reaction time: 1 hour under reflux

A further object of the present invention is a new intermediate for the manufacture of the compound 4-[(Z)-[[4-[(dimethylamino)methyl]phenyl]amino](6-fluoro-1,2-dihydro-2-oxo-3H-indol-3-ylidene)methyl]-benzenepropanoic acid, namely the compound 4-[(E)-(6-fluoro-1,2-dihydro-2-oxo-3H-indol-3-ylidene)hydroxymethyl]-benzenepropanoic acid, ethyl ester. The chemical formula of this compound is depicted below as Formula II.

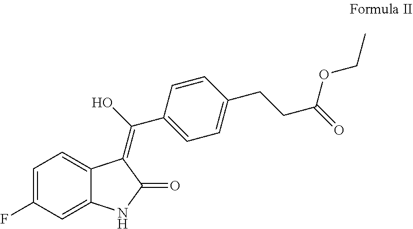

### DETAILED DESCRIPTION OF THE INVENTION

In the following, the experimental details of the synthesis are described via examples.

The following starting compounds and reagents are all commercially available.
6-Fluoro-oxindole (6-fluoro-2-indolinone), CAS 56341-39-0, is commercially available.
2,5-difluoronitrobenzene, CAS 364-74-9, for the synthetic route described in WO 04/009547 in Example I-IV is commercially available.
4-carboxybenzaldehyde, CAS 619-66-9, used for the synthesis of 4-(2-ethoxycarbonylethyl)benzoic acid (preparation analogously to Tetrahedron 1997, 53, 7335-7340) is commercially available.
4-amino-N,N-dimethyl-benzenemethanamine, CAS 6406-74-2, is commercially available.

### Example 1

### Process for the synthesis of the compound 4-[(Z)-[[4-[(dimethylamino)methyl]phenyl]amino](6-fluoro-1,2-dihydro-2-oxo-3H-indol-3-ylidene)methyl]-benzenepropanoic acid as depicted in Scheme 1 above

### Synthesis Step 1

### Synthesis of 4-[(E)-(1-acetyl-6-fluoro-1,2-dihydro-2-oxo-3H-indol-3-ylidene)-hydroxymethyl]-benzenepropanoic, acid ethyl ester

This synthesis step is described in WO 04/009547, under Example 10.1 and using the starting material of Example VI.22.

4-[(E)-(1-acetyl-6-fluoro-1,2-dihydro-2-oxo-3H-indol-3-ylidene)-hydroxymethyl]-benzenepropanoic acid, ethyl ester, or 1-acetyl-341-hydroxy-1-(4-(2-ethoxycarbonylethyl)phenyl)methylene]-6-fluoro-2-indolinone is prepared from 1-acetyl-6-fluoro-2-indolinone (described in WO 04/009547, under Example V) and 4-(2-ethoxycarbonylethyl)benzoic acid (preparation analogously to Tetrahedron 1997, 53, 7335-7340).

### Synthesis Step 2

### Synthesis of 4-[(E)-(6-fluoro-1,2-dihydro-2-oxo-3H-indol-3-ylidene)-hydroxymethyl]-benzenepropanoic acid, ethyl ester

1.62 kg (4.077 mol) 4-[(E)-(1-acetyl-6-fluoro-1,2-dihydro-2-oxo-3H-indol-3-ylidene)hydroxymethyl]-benzenepropanoic acid, ethyl ester are suspended in 14 L methanol, and 220 g (3.873 mol) sodium methoxide are added. After stirring for 1 hour under reflux the solution is cooled to 15° C. 340 ml (4.079 mol) hydrochloride acid 37% in 3.7 L water is added at 15° C. The obtained precipitate is suction filtered, washed with 8 litres of water/methanol in proportion 1:1 and dried at 60° C.

Yield: 1.29 kg (89% of theory)

T_{m.p}=163° C. (DSC 10K/min)

Purity according to HPLC: 95.2% (column: Prontosil 120-3-C18, 3 μm)

Empirical formula: C₂₀H₁₈FNO₄

ESI mass spectrum: m/z=356 [M+H]⁺

### Synthesis Step 3

### Synthesis of 4-[(Z)-[[4-[(dimethylamino)methyl]phenyl]amino](6-fluoro-1,2-dihydro-2-oxo-3H-indol-3-ylidene)methyl]-benzenepropanoic acid, ethyl ester

3.07 kg (4.444 mol) 4-[(E)-(6-fluoro-1,2-dihydro-2-oxo-3H-indol-3-ylidene)hydroxymethyl]-benzenepropanoic acid, ethyl ester are suspended in 7.0 L dioxane. After addition of 1100 ml (8.639 mol) trimethylsilylchloride and 1.363 kg (9.071) 4-amino-N,N-dimethyl-benzenemethanamine, the temperature is raised up to about 30° C. 3.65 L (17.278 mol) hexamethyldisilazane and 4.2 L dioxane are added. The mixture is heated to about 100° C. and stirred for about 60 hours. After cooling to about 60° C. and carefully addition of 12 L ethanol the solvents are evaporated under vacuum. The residue is dissolved in 10 L ethanol under reflux. The solution is cooled to about 8° C. and the obtained precipitate is suction filtered, washed with 3.2 litres of ethanol and dried at 45° C. under vacuum.

Yield: 3.355 kg (79.7% of theory)

T_{m.p}=159° C. (DSC 10K/min)

Purity according to HPLC: 99.1% (column: Prontosil 120-3-C18, 3 μm)

Empirical formula: C₂₉H₃₀FN₃O₃

ESI mass spectrum: m/z=488 [M+H]⁺

### Synthesis step 4

### Synthesis of 4-[(Z)-[[4-[(dimethylamino)methyl]phenyl]amino](6-fluoro-1,2-dihydro-2-oxo-3H-indol-3-ylidene)methyl]-benzenepropanoic acid

1055 g (2.164 mol) of 4-[(Z)-[[4-[(dimethylamino)methyl]phenyl]amino](6-fluoro-1,2-dihydro-2-oxo-3H-indol-3-ylidene)methyl]-benzenepropanoic acid, ethyl ester are suspended in 8.9 L of methanol. 4330 ml of 1 mol/l sodium hydroxide solution are added and the mixture is heated to about 70° C. After stirring for another two hours at about 70° C. the solution is cooled to about 20° C. 2200 ml of 1 mol/l hydrochloride acid is added, the yellow precipitate formed is suction filtered and washed with water. The substance is dried under vacuum at 55° C.

Yield: 939 g (94.4% of theory),

T_{m.p}=176° C.

Empirical formula: C₂₇H₂₆FN₃O₃

ESI mass spectrum: m/z=460 [M+H]⁺

Water content: 2.5% (KF) direct after drying

6-10% (KF) after equlibration on air

### Example 2

### Alternative process for the synthesis of the compound 4-[(Z)-[[4-[(dimethylamino)methyl]phenyl]amino](6-fluoro-1,2-dihydro-2-oxo-3H-indol-3-ylidene)methyl]-benzenepropanoic acid as depicted in above Scheme 2

### Synthesis step 1

### Synthesis of 4-[(E)-(1-acetyl-6-fluoro-1,2-dihydro-2-oxo-3H-indol-3-ylidene)-hydroxymethyl]-benzenepropanoic acid, ethyl ester

A solution of 2.127 kg (11.01 mol) 1-acetyl-6-fluoro-1,3-dihydro-2H-indol-2-one (or 1-acetyl-6-fluoro-2-indolinone, as described in WO 04/009547 under Example V), 100 g (0.819 mol) 4-dimethylaminopyridine and 3.368 L (24.294 mol) triethylamine in 12 L dichloromethane is cooled to 5° C. A solution of 2.923 kg (12.147 mol) 3-(4-chlorocarbonyl-phenyl) propionic acid ethyl ester, synthesised from 4-carboxy-benzenepropanoic acid, α-ethyl ester with thionylchloride in toluene (preparation analogously to Tetrahedron 1997, 53, 7335-7340), is added during 2 hours. After stirring for another 2 hours the suspension is added to 15 L hydrochloride acid 2 mol/L, the dark organic phase is separated and evaporated to dryness. The residue is dissolved in 12 L methanol, cooled to 0° C. and the obtained precipitate is suction filtered, washed with 4 litres of cold methanol and dried at 40° C.

Yield: 3.175 kg (72.6% of theory)

T_{m.p}=64° C. (DSC 10K/min)

Purity according to HPLC: 89.3% (column: Prontosil 120-3-C18, 3 μm)

Empirical formula: C₂₂H₂₀FNO₅

ESI mass spectrum: m/z=398 [M+H]⁺

### Synthesis Step 2

### Synthesis of 4-[(E)-(6-fluoro-1,2-dihydro-2-oxo-3H-indol-3-ylidene)-hydroxymethyl]-benzenepropanoic acid, ethyl ester

1.62 kg (4.077 mol) benzenepropanoic acid, 4-[(E)-(1-acetyl-6-fluoro-1,2-dihydro-2-oxo-3H-indol-3-ylidene)hydroxymethyl]-, ethyl ester are suspended in 14 L methanol, and 220 g (3.873 mol) sodium methoxide are added. After stirring for 1 hour under reflux the solution is cooled to 15° C. 340 ml (4.079 mol) hydrochloride acid 37% in 3.7 L water is added at 15° C. The obtained precipitate is suction filtered, washed with 8 litres of water/methanol in proportion 1:1 and dried at 60° C.

Yield: 1.29 kg (89% of theory)

T_{m.p}=163° C. (DSC 10K/min)

Purity according to HPLC: 95.2% (column: Prontosil 120-3-C18, 3 μm)

Empirical formula: C₂₀H₁₈FNO₄

ESI mass spectrum: m/z=356 [M+H]⁺

### Synthesis Step 3

### Synthesis of 4-[(Z)-[[4-(dimethylamino)methyl]phenyl]amino](6-fluoro-1,2-dihydro-2-oxo-3H-indol-3-ylidene)methyl]-benzenepropanoic acid, ethyl ester

3.07 kg (4.444 mol) 4-[(E)-(6-fluoro-1,2-dihydro-2-oxo-3H-indol-3-ylidene)hydroxymethyl]-benzenepropanoic acid, ethyl ester are suspended in 7.0 L dioxane. After addition of 1100 ml (8.639 mol) trimethylsilylchloride and 1.363 kg (9.071) 4-amino-N,N-dimethyl-benzenemethanamine, the temperature is raised up to about 30° C. 3.65 L (17.278 mol) hexamethyldisilazane (HMDS) and 4.2 L dioxane are added. The mixture is heated to about 100° C. and stirred for about 60 hours. After cooling to about 60° C. and carefully addition of 12 L ethanol the solvents are evaporated under vacuum. The residue is dissolved in 10 L ethanol under reflux. The solution is cooled to about 8° C. and the obtained precipitate is suction filtered, washed with 3.2 litres of ethanol and dried at 45° C. under vacuum.

Yield: 3.355 kg (79.7% of theory)

T_{m.p}=159° C. (DSC 10K/min)

Purity according to HPLC: 99.1% (column: Prontosil 120-3-C18, 3 μm)

Empirical formula: C₂₉H₃₀FN₃O₃

ESI mass spectrum: m/z=488 [M+H]⁺

### Synthesis Step 4

### Synthesis of 4-[(Z)-[[4-[(dimethylamino)methyl]phenyl]amino](6-fluoro-1,2-dihydro-2-oxo-3H-indol-3-ylidene)methyl]-benzenepropanoic acid

1055 g (2.164 mol) of 4-[(Z)-[[4-[(dimethylamino)methyl]phenyl]amino](6-fluoro-1,2-dihydro-2-oxo-3H-indol-3-ylidene)methyl]-benzenepropanoic acid, ethyl ester are suspended in 8.9 L of methanol. 4330 ml of 1 mol/l sodium hydroxide solution are added and the mixture is heated to about 70° C. After stirring for another two hours at about 70° C. the solution is cooled to about 20° C. 2200 ml of 1 mol/l hydrochloride acid is added, the yellow precipitate formed is suction filtered and washed with water. The substance is dried under vacuum at 55° C.

Yield: 939 g (94.4% of theory),

T_{m.p}=176° C.

Empirical formula: C₂₇H₂₆F N₃O₃

ESI mass spectrum: m/z=460 [M+H]⁺

Water content: 2.5% (KF) direct after drying
6-10% (KF) after equlibration on air

### Example 3

### Process for the synthesis of the compound 4-[(Z)-[[4-[(dimethylamino)methyl]phenyl]amino](6-fluoro-1,2-dihydro-2-oxo-3H-indol-3-ylidene)methyl]-benzenepropanoic acid as depicted in above Scheme 3

### Synthesis Step 1 is as Described Above in Example 2

### Synthesis Steps 2 and 3 (Performed in One Step)

### Synthesis of 4-[(Z)-[[4-(dimethylamino)methyl]phenyl]amino](6-fluoro-1,2-dihydro-2-oxo-3H-indol-3-ylidene)methyl]-benzenepropanoic acid, ethyl ester

5.0 g (12.58 mmol) 4-[(E)-(1-acetyl-6-fluoro-1,2-dihydro-2-oxo-3H-indol-3-ylidene)hydroxymethyl]-benzenepropanoic acid, ethyl ester, 3.5 g (18.87 mmol) 4-amino-N,N-dimethyl-benzenemethanamine, and 0.1 g p-toluenesulfonic acid monohydrate are suspended in 20 ml hexamethyldisilazane (HMDS). The mixture is heated to about 120° C. and stirred for 3 hours. After cooling to about 20° C. and carefully addition of 20 ml methanol 0.1 g sodium methoxide is added and the suspension is stirred for 2 hours. The precipitate is suction filtered, washed with 5 ml of methanol and dried at 45° C. under vacuum.

Yield: 2.7 g (44% of theory)

Empirical formula: C₂₉H₃₀FN₃O₃

ESI mass spectrum: m/z=488 [M+H]⁺

Synthesis step 4 is as described above in examples 1 or 2.

### Example 4

### Process for the synthesis of the compound 4-[(Z)-[[4-[(dimethylamino)methyl]phenyl]amino](6-fluoro-1,2-dihydro-2-oxo-3H-indol-3-ylidene)methyl]-benzenepropanoic acid as depicted in above Scheme 3

### Synthesis Step 1 is as Described Above in Example 2

### Synthesis step 2

### Synthesis of 4-[(Z)-[[4-[(dimethylamino)methyl]phenyl]amino](1-acetyl-6-fluoro-1,2-dihydro-2-oxo-3H-indol-3-ylidene)methyl]-benzenepropanoic acid, ethyl ester

27 g (64.9 mmol) 4-[(E/Z)-(1-acetyl-6-fluoro-1,2-dihydro-2-oxo-3H-indol-3-ylidene)chloro methyl]-benzenepropanoic acid, ethyl ester, 14.6 g (78.1 mmol) 4-amino-N,N-dimethyl-benzenemethanamine hydrochloride and 18.9 ml (136.3 mmol) triethylamine are suspended in 540 mL tetrahydrofurane and refluxed under stirring for 2 days. After evaporation of the solvent the residue is dissolved in ethylacetate/water. The organic phase is evaporated to dryness, the residue is solved in 100 ml diisopropylether/ethanol by heating, the solution is cooled to 15° C., the obtained precipitate is suction filtered and dried at 40° C. under vacuum.

Yield: 20.9 g (61% of theory)

Empirical formula: C₃₁H₃₂FN₃O₄

ESI mass spectrum: m/z=530 [M+H]⁺

### Synthesis Steps 3 and 4 (Performed in One Step)

### Synthesis of 4-[(Z)-[[4-[(dimethylamino)methyl]phenyl]amino](6-fluoro-1,2-dihydro-2-oxo-3H-indol-3-ylidene)methyl]benzenepropanoic acid

22.7 g (42.86 mmol) of 4-[(Z)-[[4-[(dimethylamino)methyl]phenyl]amino](1-acetyl-6-fluoro-1,2-dihydro-2-oxo-3H-indol-3-ylidene)methyl]-benzenepropanoic acid, ethyl ester are suspended in 227 ml of methanol. 90.8 ml of 1 mol/l sodium hydroxide solution are added and the mixture is heated to about 70° C. After stirring for another two hours at about 70° C. the solution is cooled to about 20° C. 52 ml of 1 mol/l hydrochloride acid is added, the yellow precipitate formed is suction filtered and washed with water. The substance is dried under vacuum at 45° C.

Yield: 17.1 g (84% of theory),

Purity according to HPLC: 99.8%

T_{m.p}=176° C.

Empirical formula: C₂₇H₂₆FN₃O₃

ESI mass spectrum: m/z=460 [M+H]⁺

Water content: 3.2% (KF)

1. Process for preparing the compound 4-(Z)-[[4-[(dimethylamino)methyl]phenyl]amino](6-fluoro-1,2-dihydro-2-oxo-3H-indol-3-ylidene)methyl]-benzenepropanoic acid as represented by Formula I 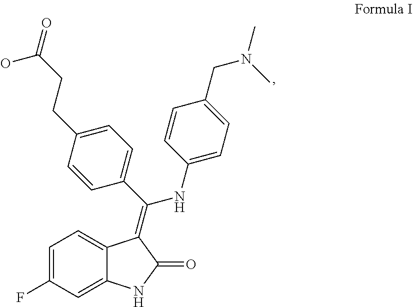
said process comprising the steps of
(a) reacting a compound of formula 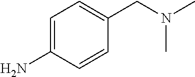
with
(i) a compound of formula 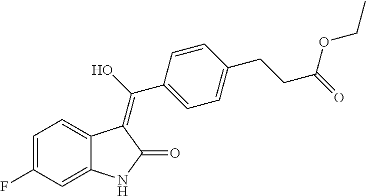
or with
(ii) a compound of formula 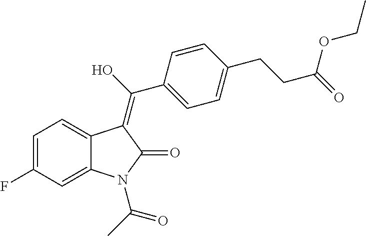
and
(b) subsequent de-esterification of the propanoic acid, ethyl ester group,
wherein the removal of the acetyl group bound to the lactame group in the compound of formula 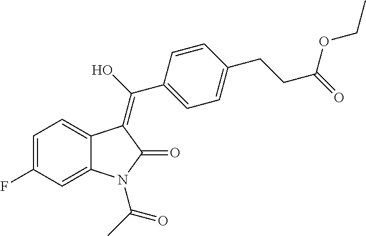
in reaction (ii) is performed after step (a),
and in which the reaction (a)(i) or (a)(ii) is performed in the presence of a mixture of reagents and solvents selected from:
Hexamethyldisilazane and p-toluenesulfonic acid monohydrate in the presence of triethylamine;
Hexamethyldisilazane and p-toluenesulfonic acid monohydrate in the presence of pyridine;
Hexamethyldisilazane and benzenesulfonic acid in the presence of triethylamine;
Hexamethyldisilazane and benzenesulfonic acid in the presence of pyridine;
Hexamethyldisilazane and trimethylsilylchloride;
N,O-bis(trimethylsily)acetamide and pyridine;
Trimethylsilylimidazolide and pyridine.

2. Process for preparing the compound 4-(Z)-[[4-[(dimethylamino)methyl]phenyl]amino](6-fluoro-1,2-dihydro-2-oxo-3H-indol-3-ylidene)methyl]-benzenepropanoic acid in accordance with claim 1, wherein said process comprising the steps of
(a) reacting a compound of formula 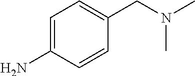
with a compound of formula 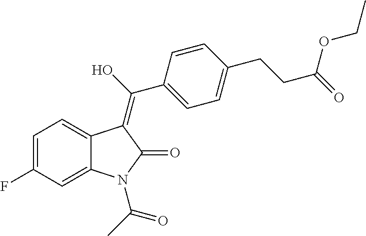
and
(b) subsequent de-esterification of the propanoic acid, ethyl ester group,
wherein the removal of the acetyl group bound to the lactame group in the compound of formula 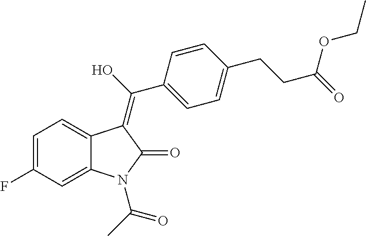
is performed after step (a),
and in which the reaction (a) is performed in the presence of a mixture of reagents and solvents selected from:
Hexamethyldisilazane and p-toluenesulfonic acid monohydrate in the presence of triethylamine;
Hexamethyldisilazane and p-toluenesulfonic acid monohydrate in the presence of pyridine;
Hexamethyldisilazane and benzenesulfonic acid in the presence of triethylamine;
Hexamethyldisilazane and benzenesulfonic acid in the presence of pyridine;
Hexamethyldisilazane and trimethylsilylchloride;
N,O-bis(trimethylsily)acetamide and pyridine;
Trimethylsilylimidazolide and pyridine.

3. Process for preparing the compound 4-(Z)-[[4-[(dimethylamino)methyl]phenyl]amino](6-fluoro-1,2-dihydro-2-oxo-3H-indol-3-ylidene)methyl]-benzenepropanoic acid in accordance with claim 1, wherein said process comprising the steps of
(a) reacting a compound of formula 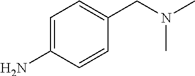
with a compound of formula 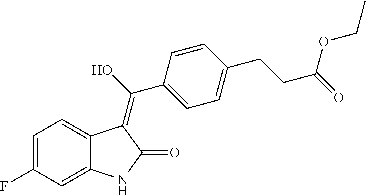
and
(b) subsequent de-esterification of the propanoic acid, ethyl ester group,
in which the reaction (a) is performed in the presence of a mixture of reagents and solvents selected from:
Hexamethyldisilazane and p-toluenesulfonic acid monohydrate in the presence of triethylamine;
Hexamethyldisilazane and p-toluenesulfonic acid monohydrate in the presence of pyridine;
Hexamethyldisilazane and benzenesulfonic acid in the presence of triethylamine;
Hexamethyldisilazane and benzenesulfonic acid in the presence of pyridine;
Hexamethyldisilazane and trimethylsilylchloride;
N,O-bis(trimethylsily)acetamide and pyridine;
Trimethylsilylimidazolide and pyridine.

4. Process for preparing the compound 4-[(Z)-[[4-[(dimethylamino)methyl]phenyl]amino](6-fluoro-1,2-dihydro-2-oxo-3H-indol-3-ylidene)methyl]-benzenepropanoic acid in accordance with claim 3, wherein the compound of formula 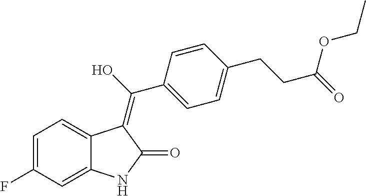
is obtained by removal of the acetyl group bound to the lactame group in a compound of formula

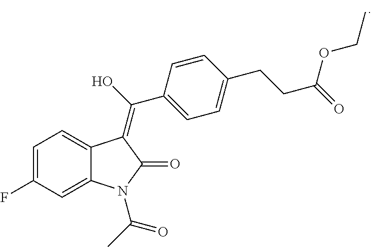

5. Process for preparing the compound 4-[(Z)-[[4-[(dimethylamino)methyl]phenyl]amino](6-fluoro-1,2-dihydro-2-oxo-3H-indol-3-ylidene)methyl]-benzenepropanoic acid in accordance with claim 4, wherein the removal of the acetyl group from the lactame group is performed in the presence of sodium methoxide.

6. Process for preparing the compound 4-[(Z)-[[4-(dimethylamino)methyl]phenyl]amino](6-fluoro-1,2-dihydro-2-oxo-3H-indol-3-ylidene)methyl]-benzenepropanoic acid in accordance with claim 2, wherein the removal of the acetyl group from the lactame group in the compound of formula 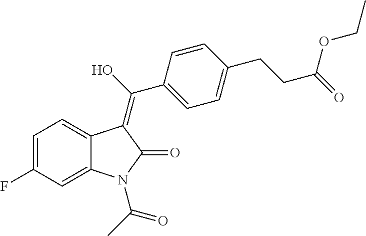
is performed by subsequent addition of methanol and sodium methoxide in the reaction medium of step (a).

7. Process for preparing 4-[(Z)-[[4-(dimethylamino)methyl]phenyl]amino](6-fluoro-1,2-dihydro-2-oxo-3H-indol-3-ylidene)methyl]-benzenepropanoic acid in accordance with claim 2, wherein the removal of the acetyl group from the lactame group in the compound of formula 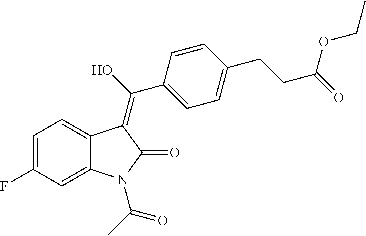
is performed in step (b).

8. Process for preparing 4-[(Z)-[[4-[(dimethylamino)methyl]phenyl]amino](6-fluoro-1,2-dihydro-2-oxo-3H-indol-3-ylidene)methyl]-benzenepropanoic acid in accordance with claim 7, wherein the removal of the acetyl group from the lactame group is performed in the same reaction medium as used for the de-esterification of the propanoic acid, ethyl ester.

9. Process for preparing the compound 4-(Z)-[[4-[(dimethylamino)methyl]phenyl]amino](6-fluoro-1,2-dihydro-2-oxo-3H-indol-3-ylidene)methyl]-benzenepropanoic acid in accordance with claim 1, wherein the compound of formula 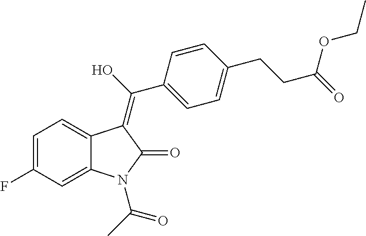
is obtained by reacting a compound of formula 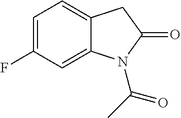
with the product of the reaction of a compound of formula 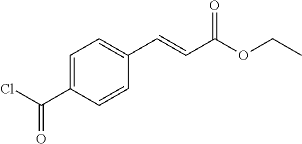
with 4-dimethylaminopyridine and triethylamine or with 4-dimethylaminopyridine and ethyldiisopropylamine.

10. Process for preparing 4-[(Z)-[[4-[(dimethylamino)methyl]phenyl]amino] (6-fluoro-1,2-dihydro-2-oxo-3H-indol-3-ylidene)methyl]-benzenepropanoic acid in accordance with claim 1, wherein the de-esterification of the propanoic acid, ethyl ester is performed by hydrolysis in the presence of sodium hydroxide.

11. The compound 4-[(E)-(6-fluoro-1,2-dihydro-2-oxo-3H-indol-3-ylidene)hydroxymethyl]-benzenepropanoic acid, ethyl ester, as represented by Formula II [chemical structure]
